(19) United States
(12) Patent Application Publication
(10) Pub. No.: US 2026/0116858 A1
(43) Pub. Date: Apr. 30, 2026

(54) NOVEL LAPPACONITINE DERIVATIVE AND USE THEREOF

(71) Applicant: QGENETICS CO., LTD., Seoul (KR)

(72) Inventors: Mun Seog CHANG, Seoul (KR); Tae Hee LEE, Daejeon (KR); Ha Young KIM, Seoul (KR); Jung Bin MIN, Seoul (KR)

(73) Assignee: QGENETICS CO., LTD., Seoul (KR)

(21) Appl. No.: 19/427,612

(22) Filed: Dec. 19, 2025

Related U.S. Application Data
(63) Continuation-in-part of application No. 17/905,797, now Pat. No. 12,516,027.

(30) Foreign Application Priority Data
Mar. 9, 2020 (KR) 10-2020-0028973

(51) Int. Cl. C07D 221/22

(52) U.S. Cl. CPC C07D 221/22

(57) ABSTRACT

The present invention pertains to a novel lappaconitine derivative and a method for producing same.

### STATEMENT OF PRIORITY

This application is a continuation-in-part of and claims priority to U.S. patent application Ser. No. 17/905,797, filed on Sep. 7, 2022, now allowed, which is a 35 U.S.C. § 371 national phase application of PCT/KR2021/002757, filed on Mar. 5, 2021, which claims priority to Korean Application No. 10-2020-0028973, filed on Mar. 9, 2020, the entire contents of each of which are incorporated by reference herein in their entirety.

### FIELD OF THE INVENTION

The present invention relates to a novel lappaconitine derivative belonging to the diterpenoid alkaloid family and a method of preparing the same.

### BACKGROUND OF THE INVENTION

Aconitine, which is one of components of Aconiti Lateralis Radix as an herbal medicine, is the most toxic chemical compound that belongs to the alkaloid family. However, when Aconiti Lateralis Radix is boiled into a decoction, it has reduced toxicity. Similarly, when heat is directly applied to such a compound, it is converted into less toxic compounds such as benzoylaconine, aconine, and the like through deacetylation or debenzoylation. Based on this principle, Aconiti Lateralis Radix (processed Aconiti Lateralis Radix or purified Aconiti Lateralis Radix) whose toxicity is reduced through the heat treatment has been used as an herbal drug to reduce inflammation and pain in patients with neuralgia (Xu et al., J Ethnopharmacol 2006).

Separately, hundreds of various alkaloid compounds, such as deoxyaconitine, mesaconitine, hypaconitine, lappaconitine, and the like, which have a similar chemical structure, and belong to the diterpenoid alkaloid family such as aconitine, have been studied through semisynthesis and structural confirmation, and their biological activity has also been elucidated (Turabekova et al., Environ Toxicol Pharmacol. 2008). For example, Shaanxi University of Science & Technology has released the results showing that lappaconitine exhibits anticancer activity by removing a 2-acetaminobenzoyl group at the carbon 4 position of lappaconitine and attaching various cinnamic derivatives thereto. The same university has also synthesized various compounds by varying the group at position 4 and functional groups at positions 8 and 9 (Liangcheng et al., CN 107540680 A, published on Jan. 5, 2018). Dr Vladimirovich (Russia) has also confirmed the inflammatory action of lappaconitine derivatives to which various aromatic compounds are attached at position 4 of lappaconitine (S. V. Vladimirovich, WO 2017/209653 A1).

Among many alkaloid compounds, lappaconitine is known to exhibit various effects such as antiarrhythmic, anti-inflammatory, antioxidant, anticancer, and antiepileptic effects (Wang et al., Chem Pharm Bull. 2009). The present inventors have prepared a compound having a new structure by hydrolyzing lappaconitine while conducting research on new pharmacological activities of lappaconitine.

### DISCLOSURE

### Technical Problem

According to one aspect of the present invention, the present invention is directed to providing a novel lappaconitine derivative and a method of preparing the same.

### Technical Solution

To achieve the objects of the present invention as described above, according to one aspect of the present invention, there is provided a compound represented by the following Formula 1, and a stereoisomer, a hydrate, a solvate, or a pharmaceutically acceptable salt thereof:

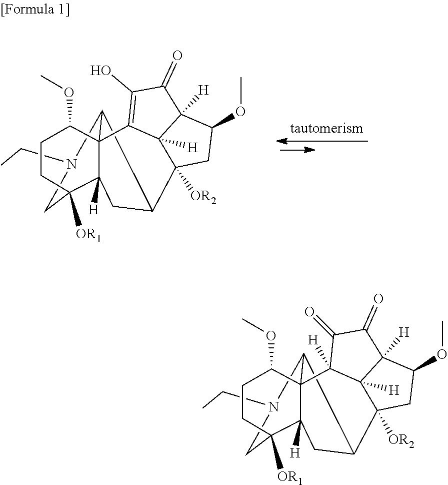

According to one embodiment of the present invention, in Formula 1 (which exists in two tautomers), R₁ and R₂ are each independently selected from the group consisting of:
H, C1-10 alkyl, C2-10 alkenyl, C3-6 cycloalkyl, C4-6 cycloalkenyl, phenyl, aromatic heterocycle, —C(O)—R₃, —C(O)—(CH₂)n-R₄, —C(S)—(CH₂)n-R₅, —C(O)—O—R₆, —C(S)—O—(CH₂)n-R₇, —C(O)—N—R8R9, —S(O)n-R₁₀ and —S(O)n-NR₁₁R₁₂;
wherein R₃ is C1-10 alkyl, C2-10 alkenyl, C3-6 cycloalkyl, C4-6 cycloalkenyl, phenyl, or an aromatic heterocycle;
in —C(O)—(CH₂)n-R₄, wherein n is 1-3, and R₄ is C1-10 alkyl, C2-10 alkenyl, C3-6 cycloalkyl, C4-6 cycloalkenyl, phenyl, or an aromatic heterocycle;
in —C(S)—(CH₂)n-R₅, wherein n is 1-3, and R₅ is C1-10 alkyl, C2-10 alkenyl, C3-6 cycloalkyl, C4-6 cycloalkenyl, phenyl, or an aromatic heterocycle;
wherein R₆ is C1-10 alkyl, C2-10 alkenyl, C3-6 cycloalkyl, C4-6 cycloalkenyl, phenyl, or an aromatic heterocycle;
in —C(S)—O—(CH₂)n-R₇, wherein n is 1-3, and R₇ is C1-10 alkyl, C2-10 alkenyl, C3-6 cycloalkyl, C4-6 cycloalkenyl, phenyl, or an aromatic heterocycle;
wherein R₈ and R₉ are each independently H, C1-10 alkyl, C2-10 alkenyl, C3-6 cycloalkyl, C4-6 cycloalkenyl, phenyl, or an aromatic heterocycle;
in —S(O)n-R₁₀, wherein n is 1 or 2, and R₁₀ is H, C1-10 alkyl, C2-10 alkenyl, C3-6 cycloalkyl, C4-6 cycloalkenyl, phenyl, or an aromatic heterocycle; and
in —S(O)n-NR₁₁R₁₂, wherein n is 1 or 2, and R₁₁ and R₁₂ are each independently H, C1-10 alkyl, C2-10 alkenyl, C3-6 cycloalkyl, C4-6 cycloalkenyl, phenyl, or an aromatic heterocycle.

According to one specific embodiment of the present invention, the compound of Chemical Formula 1 has the structure represented by Formula 2. At equilibrium, two tautomers are possible, and the compound predominantly exists in an enol-ketone form.

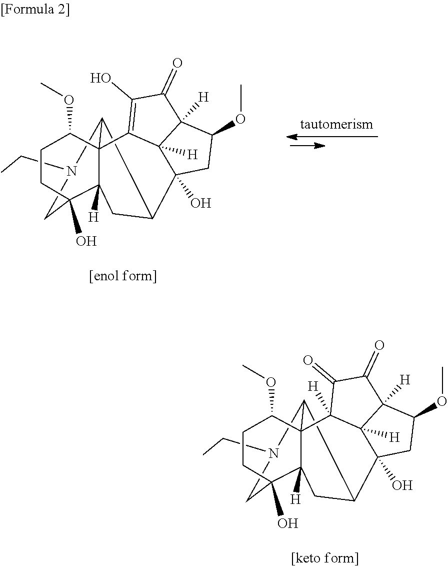

In the present invention, the compound of Formula 1 includes a hydrate, a solvate, stereoisomer, and a radioactive derivative thereof, as well as the compound represented by Formula 1 and a pharmaceutically acceptable salt thereof.

The term “pharmaceutically acceptable salt” refers to a salt that is suitable for use in contact with the tissue from humans and lower animals without causing excessive toxicity, stimulations, allergic reactions, and the like within the scope of sound medical judgment and does not have an adverse effect on the biological activity and physicochemical properties of a parental compound. The pharmaceutically acceptable salt is well known in the art. For example, the pharmaceutically acceptable salts are described in detail in S. M. Berge et al., J. Pharmaceutical Sciences, 66, 1, 1977. The salt may be prepared in situ while finally separating and purifying the compound of the present invention, or separately prepared through a reaction with an inorganic base or an organic base. For example, suitable addition salt forms include ammonium salts; alkali metal salts such as salts of lithium, sodium, potassium, magnesium, calcium, and the like, and alkaline earth metal salts (calcium salt, and the like); salts with organic bases, for example, primary, secondary, and tertiary aliphatic and aromatic amines (such as methylamine, ethylamine, propylamine, isopropylamine), quaternary butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, benzathine, N-methyl-D-glucamine, 2-amino-2-(hydroxymethyl)-1,3-propanediol, hydrabamine salt; and salts with amino acids such as arginine, lysine, and the like.

In the present invention, the hydrate or solvate of the compound of Formula 1 may be prepared using a conventional method, for example prepared by dissolving the base compound of Formula 1 in a solvent such as water, methanol, ethanol, acetone, 1,4-dioxane, adding a free acid or a free base thereto, and crystallizing or recrystallizing the resulting mixture.

Also, the compound of Formula 1 may have one or more asymmetric centers. In this case, an enantiomer or a diastereoisomer may be present for such a compound. Therefore, the compound of the present invention includes either an enantiomer or a diastereoisomer or an isomer mixture thereof. Also, the different isomers may be separated or decomposed using a conventional method, or any isomers may be obtained by a conventional synthesis method or through stereospecific or asymmetric synthesis. In addition, the compound of the present invention includes radioactive derivatives of the compound represented by Formula 1, and these radioactive compounds are useful in the field of biological research.

In the present invention, the compound of Formula 1 may be prepared using a method which includes the following steps:
(a) allowing lappaconitine to react with an oxidizing agent; and
(b) allowing the reaction product of (a) to react with an organic solvent in the presence of a base.

According to one embodiment of the present invention, the lappaconitine of (a) may be lappaconitine hydrogen bromide. In this case, the step (a) may further include removing hydrogen bromide before allowing the lappaconitine to react with the oxidizing agent. For example, the removing of the hydrogen bromide from the lappaconitine hydrogen bromide may be performed using dichloromethane (CH₂Cl₂) in the presence of a base as described in the following Scheme 1.

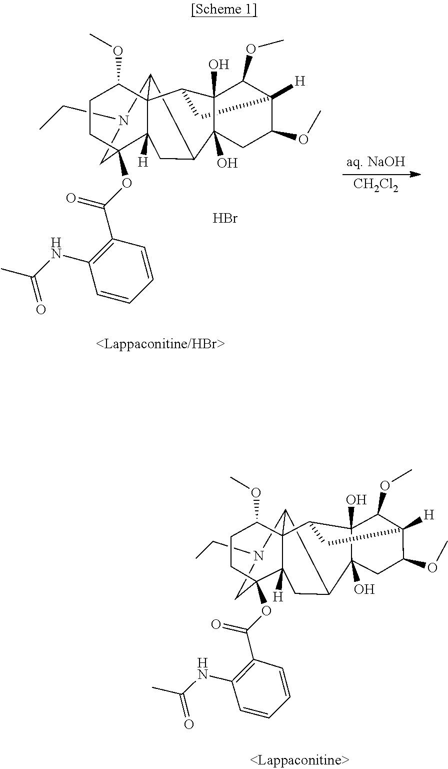

Next, the lappaconitine may be oxidized through reaction with an oxidizing agent, as shown in the following Scheme 2, thereby generating a lappaconitine derivative (LAD). The oxidizing agent may be selected from the group consisting of phenyliodine diacetate PhI(OAc)₂ or lead (II) acetate [Pb(CH₃CO₂)₂], lead (IV) acetate [Pb(CH₃CO₂)₄], ozone, and HIO₄, each of which is dissolved in dimethylformamide (DMF).

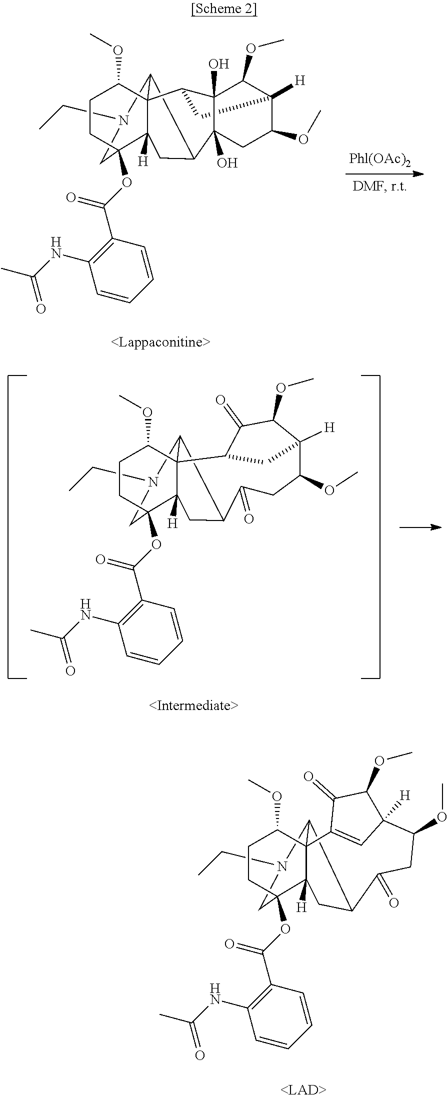

In the present invention, the synthesized lappaconitine derivative (LAD) may be allowed to react with an organic solvent in the presence of a base in order to generate the compound of Formula 1. The base may be selected from the group consisting of sodium hydroxide, potassium carbonate, sodium carbonate, cesium carbonate, and potassium hydroxide, and the organic solvent may be an aliphatic alcohol or an alkoxy alcohol.

The aliphatic alcohol refers to an alcohol represented by the general formula: CH₃(CH₂)nOH (wherein n is 0 or a positive integer), and the alkoxy alcohol refers to an alcohol represented by the general formula: CH₃(CH₂)nO(CH₂)nCH₃ (wherein n is each independently 0 or a positive integer). For example, the aliphatic alcohol may be specifically methanol, ethanol, n-propanol, isopropanol, n-butanol, and the like, and the alkoxy alcohol may be methoxymethanol, methoxyethanol, ethoxyethanol, and the like.

For example, the lappaconitine derivative (LAD) may be allowed to react with ethanol in the presence of sodium hydroxide to generate the compound (QG3030) of Formula 1, as shown in the following Scheme 3.

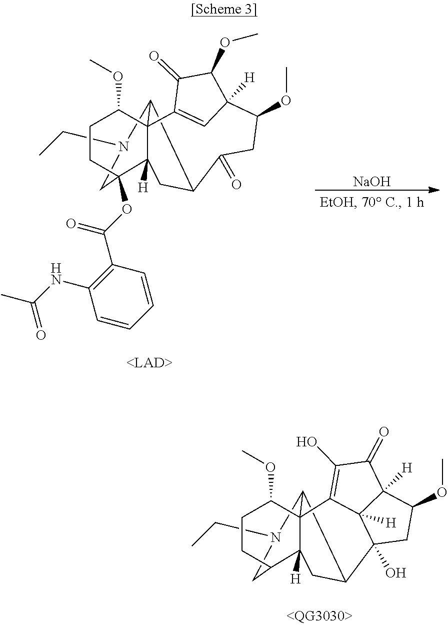

In the present invention, the compound of Formula 1 synthesized by the method may be separated using typical separation and purification processes, for example, by diluting the mixture in an organic solvent, washing the mixture, and concentrating an organic layer under reduced pressure. When necessary, the compound of Formula 1 may be purified by column chromatography and a recrystallization method using various solvents.

### Advantageous Effects

The present invention provides a lappaconitine derivative and method of preparing the same.

### DESCRIPTION OF DRAWINGS

FIG. 1 shows a structure of a lappaconitine derivative (QG3030) according to one embodiment of the present invention.

FIG. 2 shows the ¹³C-NMR data of QG3030.

FIG. 3 shows the results of measuring the total energies of the tautomeric forms of QG3030.

### BEST MODE

Hereinafter, one or more embodiments of the present invention will be described in detail with reference to examples thereof. However, it should be understood that the examples are for exemplary illustration and are not intended to limit the scope of the present invention.

### Preparation Example: Preparation of QG3030

### 1-1. Preparation of Lappaconitine

Dichloromethane (500 mL) and a sodium hydroxide solution (10 g of NaOH, and 100 g of water) were added to lappaconitine hydrogen bromide (10.0 g, 0.015 mol), and an organic layer and an aqueous layer were separated using a separatory funnel. The separated organic layer was washed several times with water, dried over anhydrous magnesium sulfate, and then distilled to obtain the target lappaconitine with a yield of 97% (8.51 g).

¹H-NMR (CDCl₃, 400 MHz) δ 11.07 (1H, s), 8.68 (1H, d, J=8.8 Hz), 7.94 (1H, d, J=6.8 Hz), 7.51 (1H, t, J=7.2 Hz), 7.04 (1H, t, J=7.9 Hz) 3.61 (1H, s), 3.58 (1H, s), 3.46 (1H, d, J=4.8 Hz), 3.42 (3H, s), 3.33 (3H, s), 3.31 (3H, s), 3.20 (1H, q, J=3.1 Hz, 7.0 Hz) 3.02 (1H, s), 2.74-2.65 (2H, m) 2.62-2.54 (2H, m), 2.53-2.48 (2H, m), 2.45-2.38 (3H, m), 2.32 (1H, s), 2.29 (1H, s), 2.24 (3H, s), 2.18 (2H, d, J=8.0 Hz), 2.13-2.09 (1H, m), 2.06-1.95 (2H, m), 1.85-1.79 (2H, m), 1.61 (1H, dd, J=6.8 Hz, 8.2 Hz), 1.14 (3H, t, J=7.2 Hz);

¹³C-NMR (CDCl₃, 100 MHz) δ 169.05, 167.40, 141.62, 134.37, 131.09, 122.33, 120.20, 115.77, 90.10, 84.63, 84.17, 82.89, 78.58, 75.57, 61.51, 57.91, 56.56, 56.12, 55.49, 50.95, 49.85, 48.99, 48.55, 47.60, 44.78, 36.26, 31.84, 26.79, 26.20, 25.55, 24.13, 13.56;

HRMS (ESI): m/z calculated for C₃₂H₄₅N₂O₈ [M+H]⁺: 585.3176.

Found 585.3170.

1-2. Preparation of Lappaconitine Derivative (LAD) from Lappaconitine

Lappaconitine (8.9 g, 0.015 mol) was slowly added to a solution in which phenyliodine diacetate (PhI(OAc)₂ (14.07 g, 0.044 mol) was dissolved in dimethylformamide (DMF, 150 mL), and stirred for 10 minutes. When the reaction was completed, the solution was diluted with ethyl acetate (EA), and extracted using an aqueous solution of saturated sodium bicarbonate (NaHCO₃). The organic layer was washed several times with water to remove dimethylformamide, dried over anhydrous magnesium sulfate, and then distilled under reduced pressure. The resulting crude product was separated by column chromatography (diethyl ether:ethyl acetate:hexane=3:2:5) to obtain {(3S,6S,7S,9S,11S,16S)-1-ethyl-6,9,11-trimethoxy-8,13-dioxododecahydro-2H-3,6a,14-(epiethane[1,1,2]triyl)-7,10-methanocyclodeca[b]azocin-3(4H)-yl 2-acetaminobenzoate} as the target lappaconitine derivative (LAD) with a yield of 46%(3.88 g).

¹H-NMR (CDCl₃, 400 MHz) δ 11.04 (1H, s), 8.68 (1H, d, J=8.4 Hz), 7.95 (1H, d, J=8.0 Hz), 7.53 (1H, t, J=7.6 Hz), 7.06 (1H, t, J=7.4 Hz), 6.98 (1H, d, J=2.8 Hz), 4.03-3.90 (3H, m), 3.81 (1H, d, J=11.2 Hz), 3.67 (3H, s), 3.48 (3H, s), 3.24 (3H, s), 2.91-2.87 (1H, m), 2.85-2.82 (1H, m), 2.71-2.62 (2H, m), 2.59-2.54 (1H, m), 2.43-2.37 (1H, m), 2.24 (3H, s), 2.17 (1H, d, J=11.6 Hz), 2.13-2.10 (1H, m), 2.04 (2H, d, J=13.2 Hz), 1.99-1.97 (2H, m), 1.81 (2H, s), 1.17 (3H, t, J=7.2 Hz);

¹³C-NMR (CDCl₃, 100 MHz) δ 211.85, 204.31, 169.10, 167.40, 162.18, 143.74, 141.74, 134.61, 131.07, 122.41, 120.30, 115.45, 86.92, 82.75, 81.45, 78.21, 76.28, 60.73, 58.17, 57.37, 54.35, 52.98, 50.19, 48.78, 46.06, 45.92, 38.41, 32.06, 25.56, 25.39, 25.36, 12.72;

HRMS (ESI): m/z calculated for C₃₂H₄₁N₂O₈ [M+H]⁺: 581.2863.

Found 581.2862.

1-3. Preparation of QG3030 from LAD

LAD (2.53 g, 0.004 mol) and NaOH (0.5 g, 0.013 mol) were dissolved in ethanol (100 mL). The temperature was then gradually increased, and the reaction mixture was stirred at 70° C. for 1 hour. Upon completion of the reaction, ethanol was removed by evaporation under reduced pressure, and the residue was adjusted to pH 10 using saturated aqueous NH₄Cl and a 15% isopropyl alcohol/CH₂Cl₂ solution (200 mL), followed by extraction. The organic layer was washed three times with distilled water, dried over anhydrous MgSO₄, and the solvent was removed under reduced pressure. The crude product was triturated (CH₂Cl₂/MeOH/hexane) to afford the final compound QG3030, and the remaining QG3030 in the residue was further purified by column chromatography (DCM/MeOH=20:1 to 10:1 gradient). QG3030 was obtained in a total yield of 80% (1.41 g).

¹H-NMR (DMSO-d₆, 400 MHz) δ 8.94 (1H, s), 4.68 (1H, s), 4.36 (1H, s), 4.24-4.18 (1H, m), 4.06-3.99 (1H, m), 3.58 (1H, s), 3.38 (3H, s), 3.18 (3H, s), 2.97 (1H, dd, J=3.6 Hz, 5.4 Hz), 2.72 (1H, d, J=5.4 Hz), 2.59 (1H, d, J=10.8 Hz), 2.55-2.53 (1H, m), 2.44-2.38 (1H, m), 2.27-2.21 (1H, m), 2.17 (1H, d, J=6.8 Hz), 1.99-1.96 (2H, m), 1.93-1.90 (1H, m), 1.78-1.75 (1H, m), 1.71-1.66 (3H, m), 1.57 (1H, t, J=12.4 Hz), 1.13-1.08 (1H, m), 1.01 (3H, t, J=7.2 Hz);

¹³C-NMR (DMSO-d₆, 100 MHz) δ 200.90, 149.54, 148.43, 82.83, 79.43, 79.10, 67.42, 62.39, 57.73, 57.18, 56.66, 52.81, 49.12, 48.47, 47.49, 46.47, 41.96, 39.50, 38.01, 26.26, 26.02, 13.21;

HRMS (ESI): m/z calculated for C₂₂H₃₂NO₆ [M+H]⁺: 406.2230.

Found 406.2224; X-ray structure.

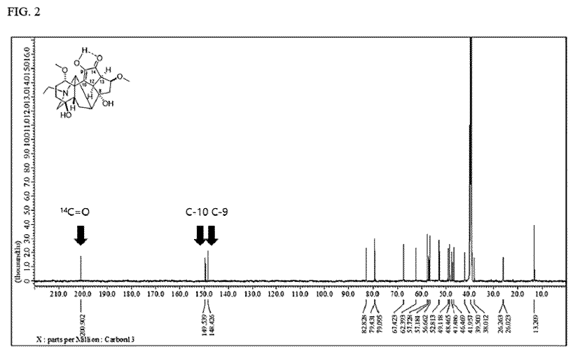

The ¹³C-NMR data of QG3030 are additionally presented in FIG. 2. Under DMSO-d₆ solvent conditions, it can be confirmed that only one C═O (ketone) group conjugated to a double bond is present, appearing at 200.9 ppm. In general, an isolated C═O (ketone) group typically resonates at around 220 ppm. In addition, two signals corresponding to the ¹⁰C═⁹C double bond were observed at 149.5 ppm and 148.4 ppm, respectively.

### 1-4. Confirmation of Structure of QG3030

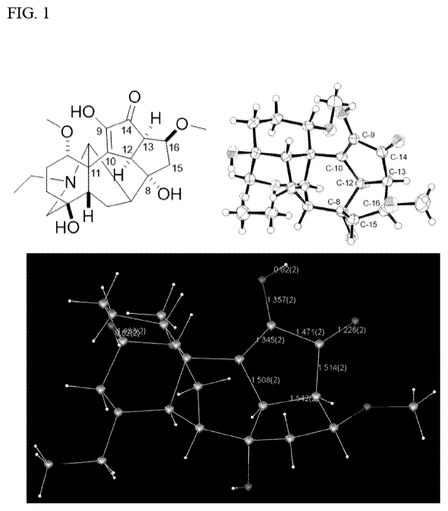

The QG3030 obtained in Preparation Example 1-3 was recrystallized with water, and appropriate crystals were obtained by X-ray diffraction. Thereafter, an X-ray structure of the crystals was determined (X-ray Diffractometer, R-AXIS RAPID). The results are shown in FIG. 1 and Table 1.

TABLE 1

| | |
|---|---|
| Molecular formula | C₂₂H₃₁NO₆ |
| Molecular weight | 405.49 |
| Crystal Color, Habit | colorless, block |
| Crystal Dimensions | 0.400 × 0.400 × 0.300 mm |
| Crystal System | orthorhombic |
| Lattice Type | primitive |
| Lattice Parameters | a = 8.9303(4) Å |
| | b = 10.6425(6) Å |
| | c = 20.7571(9) Å |
| | V = 1972.76(17) Å3 |
| Space Group | P2₁2₁2₁ (#19) |
| Z value | 4 |
| Dcalc | 1.365 g/cm³ |
| F000 | 872.00 |
| μ(MoKa) | 0.986 cm⁻¹ |

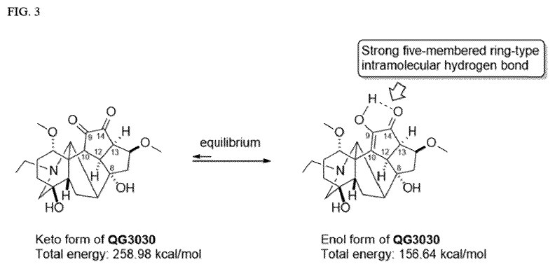

In addition, the total energies of the tautomers of QG3030 were evaluated (FIG. 3; nonpolar hydrogen atoms are omitted). Based on Equation [1] below, it can be concluded that the more stable enol form exists as the predominant tautomer.

$$Δ G = - RT ln K\_eq = - 1 \; 02 kcal / mol ( Boltzmann distribution ) \qquad [ Equation 1 ]$$

wherein K_eq=[enol form]/[keto form].

Specifically, the enol form is more stable due to the presence of a strong intramolecular hydrogen bond and a conjugated system formed by the α, β-unsaturated ketone moiety.

What is claimed is:

1. A compound represented by the following Formula 1, and a stereoisomer, a hydrate, a solvate, or a pharmaceutically acceptable salt thereof: 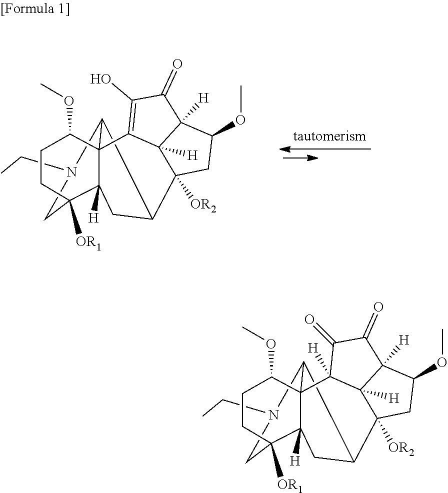
wherein R₁ and R₂ are each independently selected from the group consisting of H, C1-10 alkyl, C2-10 alkenyl, C3-6 cycloalkyl, C4-6 cycloalkenyl, phenyl, aromatic heterocycle, —C(O)—R₃, —C(O)—(CH₂)n-R₄, —C(S)—(CH₂)n-R₅, —C(O)—O—R₆, —C(S)—O—(CH₂)n-R₇, —C(O)—N—R8R9, —S(O)n-R₁₀ and —S(O)n-NR₁₁R₁₂;
wherein R₃ is C1-10 alkyl, C2-10 alkenyl, C3-6 cycloalkyl, C4-6 cycloalkenyl, phenyl, or an aromatic heterocycle;
in —C(O)—(CH₂)n-R₄, wherein n is 1-3, and R₄ is C1-10 alkyl, C2-10 alkenyl, C3-6 cycloalkyl, C4-6 cycloalkenyl, phenyl, or an aromatic heterocycle;
in —C(S)—(CH₂)n-R₅, wherein n is 1-3, and R₅ is C1-10 alkyl, C2-10 alkenyl, C3-6 cycloalkyl, C4-6 cycloalkenyl, phenyl, or an aromatic heterocycle;
wherein R₆ is C1-10 alkyl, C2-10 alkenyl, C3-6 cycloalkyl, C4-6 cycloalkenyl, phenyl, or an aromatic heterocycle;
in —C(S)—O—(CH₂)n-R₇, wherein n is 1-3, and R₇ is C1-10 alkyl, C2-10 alkenyl, C3-6 cycloalkyl, C4-6 cycloalkenyl, phenyl, or an aromatic heterocycle;
wherein R₈ and R₉ are each independently H, C1-10 alkyl, C2-10 alkenyl, C3-6 cycloalkyl, C4-6 cycloalkenyl, phenyl, or an aromatic heterocycle;
in —S(O)n-R₁₀, wherein n is 1 or 2, and R₁₀ is H, C1-10 alkyl, C2-10 alkenyl, C3-6 cycloalkyl, C4-6 cycloalkenyl, phenyl, or an aromatic heterocycle; and
in —S(O)n-NR₁₁R₁₂, wherein n is 1 or 2, and R₁₁ and R₁₂ are each independently H, C1-10 alkyl, C2-10 alkenyl, C3-6 cycloalkyl, C4-6 cycloalkenyl, phenyl, or an aromatic heterocycle.

2. The compound represented by the following Formula 1, and a stereoisomer, a hydrate, a solvate, or a pharmaceutically acceptable salt as claimed in claim 1, wherein R₁ and R₂ are each independently hydrogen.

3. A method of synthesizing the compound of claim 1, comprising:
(a) allowing lappaconitine to react with an oxidizing agent; and
(b) allowing the reaction product of (a) to react with an organic solvent in the presence of a base.

4. The method of claim 3, wherein the oxidizing agent of (a) is selected from the group consisting of phenyliodine diacetate PhI(OAc)₂, lead (II) acetate [Pb(CH3CO₂)₂], lead (IV) acetate [Pb(CH₃CO₂)₄], ozone, and HIO₄.

5. The method of claim 3, wherein the base of (b) is selected from the group consisting of sodium hydroxide, potassium carbonate, sodium carbonate, cesium carbonate, and potassium hydroxide.

6. The method of claim 3, wherein the organic solvent of (b) is an aliphatic alcohol or an alkoxy alcohol.
